(19) United States
(12) Patent Application Publication
(10) Pub. No.: US 2015/0174193 A1
(43) Pub. Date: Jun. 25, 2015

(54) AGENTS FOR TREATING CYSTIC FIBROSIS

(71) Applicants: INSERM, Paris (FR); UNIVERSITE DE TOURS FRANCOIS RABELAIS, Tours (FR)

(72) Inventors: Patrick Midoux, Orleans (FR); Francis Gauthier, Tours (FR); Sylvie Attucci, Tours (FR); Virginie Herve, Tours (FR); Alice Dubois, Tours (FR)

(21) Appl. No.: 14/403,693

(22) PCT Filed: May 24, 2013
(86) PCT No.: PCT/EP2013/060799

§ 371 Date: Nov. 25, 2014

(30) Foreign Application Priority Data
May 25, 2012 (EP) 12169547.2

(51) Int. Cl. A61K 38/02; A61K 45/06

(52) U.S. Cl. CPC A61K 38/02; A61K 45/06

(57) ABSTRACT

The present invention relates to compositions comprising cationic polymers and their use in treating inflammatory lung diseases characterized by the recruitment of blood neutrophils in the airways favoring the formation of a thick, mucoid or mucopurulent sputum as found in cystic fibrosis.

The present invention relates to compositions comprising cationic polymers and their use in treating cystic fibrosis, or other inflammatory lung disorders characterized by the recruitment of blood neutrophils in the airways favoring the formation of a thick, mucoid or mucopurulent sputum.

Cystic fibrosis and other lung disorders are characterized by obstruction of the airways caused by the accumulations of viscous secretions. In particular, cystic fibrosis (CF) owes its morbidity and mortality primarily to the devastating effects of chronic inflammation and infection within the pulmonary airway.

The opportunistic bacteria P aeruginosa and S aureus are most often present in the lung secretions of patients with cystic fibrosis (CF), where they contribute to maintain chronic inflammation in the lung (1). The presence of thick and sticky sputum that characterizes CF secretions greatly impairs mucociliary clearance and thus the elimination of these pathogens. DNA is a major component of CF sputum that contributes to airways obstruction and to innate immune response failure observed during this disease (2). Thus it favors infection and colonization by these opportunistic bacteria.

Most of the DNA present in CF expectorations is released from dead neutrophils or is actively secreted as neutrophil extracellular traps (NETs) by activated neutrophils. NETs have been reported as antimicrobial weapons made of chromatin fibers covered with granular antimicrobial proteins and proteases that trap and kill pathogens (3). It was recently assumed that NETs formation would represent the major factor underlying the gel-like structure of CF sputum (4). The nebulization in CF airways of recombinant human DNase reduces the viscoelasticity of CF sputum and enhances the clearance of secretions, which illustrates the essential contribution of DNA to the physical properties of CF lung secretions. Though DNase is the most widely used mucoactive agent for CF patients, sputum fluidization may also be obtained using hypertonic saline which increases the volume of airway surface liquid and restores mucus clearance (5, 6).

Another hallmark of cystic fibrosis is the presence in lung secretions of an uncontrolled proteolytic potential mainly due to neutrophil serine proteases (NSPs). Treatment of cystic fibrosis patients with exogenous protease inhibitors such as α1-anti-trypsin, or secretory leukoprotease inhibitor have also been proposed (7). The inventors have recently shown that NSPs, i.e. neutrophil elastase, protease 3 and cathepsin G in CF sputum are active and lack sensitivity to exogenous protease inhibitors (8). Elastase activity in a CF sputum homogenate increases dramatically after DNase treatment but also is far better controlled by exogenous protease inhibitors. The inventors hypothesized that this results from the release of DNA-bound elastase that gains in activity and/or becomes more accessible to protease inhibitors once solubilized. However, no such increase in activity is observed with the other two NSPs, probably because of their tighter binding to DNA or to their binding to other macromolecular components present in sputum.

Conventional treatments of CF include secretion clearance, antibiotic treatments, anti-inflammatory treatments.

It was known in the Art that cationic polymers and in particular poly-L-lysine have the capacity to form electrostatic complexes with DNA and to induce its condensation as aggregates. Such cationic polymers are in development as gene therapy non-viral vectors (9) including for the treatment of cystic fibrosis (http://www.worldscibooks.com/lifesci/7406.html). Cationic polymers have also been described as inhibitors of mucin secretion from airway goblet cells in vitro, and suggested to be used as a tool in the treatment of diseases associated with mucin hypersecretion (U.S. Pat. No. 6,245,320).

α-poly-L-lysine and positively charged amino acids have also been described in the art for their anti-microbial properties (10).

The inventors have now found that cationic polymers such as poly-L-lysine have the following properties:

i/ they condensate DNA, and in particular extracellular DNA from neutrophil extracellular traps (NETs) in CF lung secretions and thus fluidize CF sputum as does recombinant human DNase;
ii/ they improve the control of extracellular proteases by exogenous inhibitors as a result of DNA condensation; i.e. they enhance anti-protease activity of protease inhibitor in cystic fibrosis sputa in vitro, and,
iii/ they act as bactericidal agents because of their cationic character.

Altogether these results provide new insights for treating cystic fibrosis, using cationic polymers either as main active principle ingredient or in combination with protease inhibitor treatment, as an adjuvant for in vivo stimulation of protease inhibition in secretions from patients suffering from an accumulation of thick, mucoid or mucopurulent sputum in the airways. Moreover, pretreatment with cationic polymers would facilitate accessibility of antibiotics and of viral and non viral vectors used for gene therapy strategies.

### SUMMARY OF THE INVENTION

Therefore, in a first aspect, the present invention provides a pharmaceutical composition comprising a cationic polymer in combination with at least a protease inhibitor.

In a specific embodiment, said cationic polymer is a polymer containing sufficient positively charged monomeric units to enable DNA condensation and said cationic polymer is present in the composition in an effective amount for enhancing anti-protease activity of said protease inhibitor in cystic fibrosis sputa in vitro, thereby allowing in vivo stimulation of protease inhibition in lung secretion from patients suffering from the accumulation in the airways of a thick, mucoid or mucopurulent sputum.

For example, said cationic polymer has at least 24 positively charged monomeric units. In a preferred embodiment, said cationic polymers are typically polyamino acid molecules with positively charged amino acid residues. Said cationic polymer may typically be poly-L-lysine, or a polyaminoacid containing at least 50%, 60%, or at least 70% of lysine. For example, said cationic polymer is a polyaminoacid molecule of lysine, optionally partially substituted with histidine, preferably of a molecular weight between 4 and 15 kDa and for example, containing between 10 and 50% histidine. The pharmaceutical compositions of the invention are preferably suitable for administration in the form of an aerosol.

They are useful for treating inflammatory lung disorders characterized by the recruitment of blood neutrophils in the airways favoring the formation of a thick, mucoid or mucopurulent sputum, such as cystic fibrosis.

In a second aspect, the invention relates to a method for treating inflammatory lung disorders characterized by the recruitment of blood neutrophils in the airways favoring the formation of a thick, mucoid or mucopurulent sputum, said method comprising aerosolizing to a subject in need thereof, a composition comprising cationic polymer(s) in an amount therapeutically effective to reduce the visco-elasticity of the sputum.

In a third aspect, the invention relates to pharmaceutical compositions, comprising such cationic polymer(s) and pharmaceutically acceptable excipient, for use in treating lung disorders characterized by the recruitment of blood neutrophils in the airways favoring the formation of a thick, mucoid or mucopurulent sputum such as cystic fibrosis.

### Cationic Polymers for Use in the Present Invention

The invention results from the discoveries of unexpected properties of certain cationic polymers to reduce the visco-elasticity of sputum present in the airways of patients suffering from disorders characterized by the recruitment of blood neutrophils in the airways favoring the formation of a thick, mucoid or mucopurulent sputum, such as cystic fibrosis.

As used herein the term “airway” refers to any part of the breathing system, including the lungs and the respiratory tract and nose.

The term “patient” refers to animals, non-human mammalian or human beings, having a pulmonary system.

Cationic polymers that can be used in the present invention may be those disclosed in the prior art for use as vector for gene therapy, in particular, non-viral gene therapy (see e.g. (9, 11)).

Examples of said cationic polymers include without limitation, poly-L-lysine, poly-L-arginine, co-poly-lysine/histidine, poly-L-lysine partially substituted with histidine, poly-L-ornithine, poly(N-vinyl imidazole-quaternized with CH₃I), branched polyethyleneimine, linear polyethyleneimine, polyethyleneimine partially substituted with histidine, poly(2-(dimethylamino)ethyl-methacrylate), Poly(amido-amine) dendrimers, chitosan, cationic cyclodextrines etc. In one specific embodiment, said cationic polymers for use according to the invention comprise a sufficient amount of positively charged monomeric units to enable DNA condensation and/or to reduce in vitro the visco-elasticity of sputum from patients suffering from cystic fibrosis. For example, said cationic polymer typically comprises at least 24 positively charged monomeric units, preferably between 30 and 72 positively charged monomeric units.

The cationic polymers are typically polyamino acid molecules with positively charged amino acid residues. In a specific embodiment, the cationic polymers for use according to the invention comprise at least 24 amino acids, for example, between 24 and 200 positively charged amino acid residues, and preferably between 24 and 72 positively charged amino acid residues. In another specific embodiment, the cationic polymers for use according to the invention has a molecular weight between 4 and 15 kDa.

Positively charged amino acid residues may be selected among any known positively charged amino acids, including without limitation lysine, histidine, arginine or ornithine. As used herein, the term “positively charged” refers to the side chain of the amino acids which has a net positive charge at a pH of 7.0. For example, said cationic polymer typically is a polyaminoacid, for example a poly-L-lysine, comprising at least 24 positively charged lysyl residues, preferably between 30 and 72 positively charged lysyl residues. Said cationic polymer may typically be poly-L-lysine, or a polyaminoacid containing at least 50%, 60%, or at least 70% of lysine. For example, said cationic polymer is a polyaminoacid molecule of lysine and histidine residues, preferably of a molecular weight between 4 and 15 kDa and possibly containing from 10 to 50% of histidine.

A cationic polymer for use according to the invention typically includes at the most 30% of the positively charged residues that could be substituted with neutral residues. For example, at the most 30% of the ε-amino group of the lysyl residues of poly-L-lysine could be substituted with neutral residues.

A cationic polymer for use according to the invention typically includes at least 50% (per monomeric unit), 60% or at least 70% of positively charged amino acid residues, preferably at least 50%, 60%, 70% (per monomeric unit) of lysine residues, for example between 50% and 90% (per monomeric unit) of lysine residues.

Parameters such as the number of monomers (e.g. number of amino acids), the type of monomeric units (e.g. type of amino acids) and the percentage of positively charged monomeric units (e.g percentage of positively charged amino acids in a polyaminoacid) may be optimized by measuring the efficacy of the final structure in an in vitro assay for assessing visco-elasticity of sputum sample from cystic fibrosis patients, as disclosed in the Examples below. At optimal concentration, the cationic polymers are capable of inducing the fractionation of the sputum in dense aggregates and a very fluid phase and/or reducing significantly the measured visco-elasticity of said sputum sample.

Preferred cationic polymers for use according to the present invention are polyamino acids, such as poly-L-lysine, poly-L-arginine, or heteropolymers of L-lysine and L-arginine, polylysine with no more than 50% of the lysyl residue substituted by histidine or neutral residue.

For example, substitution of lysyl residues of poly-L-lysine with histidine is described in (Midoux, P. and Monsigny, M. (1999). Efficient gene transfer by histidylated polylysine/pDNA complexes. Bioconjugate Chem. 10, 406-411).

In specific embodiments, the cationic polymers may be partially substituted with histidine residues in order to decrease the cytotoxicity. For example, substitution of linear polyethyleneimine with histidine is described in (Bertrand, E., Gonçalves, C., Billiet, L., Gomez, J-P., Pichon, C., Cheradame, H., Midoux, P. and Guégan, P. (2011) Histidinylated linear PEI: a new efficient non toxic polymer for gene transfer. Chem. Commun., 47: 12547-12549.)

Equivalent amino acids may also be used in the cationic polymers of the invention, including amino acids having side chain modifications or substitutions, the final polymer retaining its advantageous property of fluidizing sputum from cystic fibrosis patients.

In particular, (D) or (L) amino acids may be used, or chemically modified amino acids, including amino acid analogs such as penicillamine (3-mercapto-D-valine), naturally occurring non-proteogenic amino acids and chemically synthesized compounds that have properties known in the art to be characteristic of an amino acid.

Cationic polymers useful for this invention can be produced using technique well known in the Art, including either chemical synthesis or recombinant DNA techniques. Cationic polypeptides can be synthesized using Solid Phase Peptide Synthesis techniques with tBoc or Fmoc protected alpha-amino acids (11). Alternatively, polycationic polypeptides can be produced using recombinant DNA techniques (See Coligan et al., Current Protocols in Immunology, Wiley Intersciences, 1991, Unit 9; U.S. Pat. No. 5,593,866).

In specific embodiments, the cationic polymers may be PEGylated. PEGylation is the process of covalent attachment of polyethylene glycol polymer chains to another molecule. Polyethylene glycol (PEG) molecules may be added onto cationic polymers in order to limit DNA complexes aggregation, adsorption of proteins and to lower aggregate as well as polymer cytotoxicity (12-14). The covalent attachment of PEG to cationic polymers may facilitate and does not compromise administration of said cationic polymers into the airways in the form of an aerosol (15).

The covalent attachment of PEG moiety onto cationic polymer can be performed by two ways leading either to a PEG-grafted-polymer or a block copolymer.

For example, PEG-grafted polylysine (PEG-g-pLK) is prepared by reaction of the N-hydroxysuccinimide derivative of the methoxypolyethylene glycol (mPEG) propionic acid (for instance of 5000 Da) with the ε-amino group of the lysyl residues of pLK (16).

For example, PEG-pLK block copolymer can be prepared either by i) reaction between equal molar ratios of pLK containing a cysteinyl residue at its C-terminal end with methoxy-PEG-maleimide as described in (17); ii) ring opening polymerization of N^{ε}-trifluoroacetyl-L-lysine N-carboxyanhydride with the ω-NH₂ terminal group of α-methoxy-ω-amino PEG as described in (18).

In other specific embodiments, the cationic polymers for use in accordance with the present invention are glycosylated. For example, substitution of lysyl residues of poly-L-lysine with mannose, galactose or lactose is described in (19, 20). Mannosyl-PEG, galactosyl-PEG or lactosyl-PEG may be grafted on poly-L-lysine as described in (21).

In other specific embodiments, the cationic polymers for use in accordance with the present invention are gluconoylated in order to decrease the number of positive charges and the cytotoxicity. For example, substitution of lysyl residues of poly-L-lysine with β-gluconolactone is described in (22).

### Pharmaceutical Formulations and Modes of Administration

In another aspect the present invention provides a composition, e.g., a pharmaceutical composition, containing the cationic polymers as described above, formulated together with a pharmaceutically acceptable carrier or excipient.

Pharmaceutical formulations comprising the cationic polymers of the invention may be prepared for storage by mixing the cationic polymers, for example poly-L-lysine polymers or their derivatives with substituted residues, having the desired degree of purity with optional physiologically acceptable carriers, excipients or stabilizers (Remington: the Science and Practice of Pharmacy 20th edition (2000)), in the form of aqueous solutions, lyophilized or other dried formulations.

Therefore, the invention further relates to a lyophilized, dried or liquid formulations comprising at least cationic polymers of the invention as described in the previous paragraph.

One remarkable finding of the present invention is that the cationic polymers are useful as active principles, and not as vector for gene therapy or carrier (as previously disclosed in the prior art). Accordingly, the present invention provides composition essentially consisting of cationic polymers of the present invention as described above, as active principles, optionally formulated with pharmaceutically acceptable carrier or excipient or stabilizers.

As used herein, ‘pharmaceutically acceptable carrier’ includes any and all solvents, dispersion media, coatings, antibacterial and antifungal agents, isotonic and absorption delaying agents, and the like that are physiologically compatible. The carrier should be suitable for inhalation, intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The cationic polymers of the invention may include one or more pharmaceutically acceptable salts. A ‘pharmaceutically acceptable salt’ refers to a salt that retains the desired biological activity of the parent compound and does not impart any undesired toxicological effects (23). Examples of such salts include acid addition salts and base addition salts. Acid addition salts include those derived from nontoxic inorganic acids, such as hydrochloric, nitric, phosphoric, sulfuric, hydrobromic hydroiodic phosphorous and the like as well as from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate. alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration.

Sterile solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile solutions, the methods of preparation are vacuum drying and freeze-drying (lyophilization) that yield a powder of the active ingredient plus any additional desired ingredient from a previously sterile-filtered solution thereof.

In a preferred embodiment, the compositions are formulated for their administration into the airways, e.g. by inhalation.

The compositions of the invention may thus be formulated as solution appropriate for inhalation. Any of the various means known in the art for administering therapeutically active agents by inhalation (pulmonary delivery) can be used in the methods of the present invention.

Such delivery methods are well-known in the art. Commercially available aerosolizers for liquid formulations, including jet nebulizers and ultrasonic nebulizers may be used. For delivery in liquid form, liquid formulation can be directly aerosolized and lyophilized powder can be aerosolized after reconstitution. For delivery in dry powder form, the formulation may be prepared as a lyophilized and milled powder. In addition, formulations may be delivered using a fluorocarbon formulation or other propellant and a metered dose dispenser.

In specific embodiments, for example, nebulizers, which convert liquids into aerosols of a size that can be inhaled into the lower respiratory tract, are used, either in conjunction with a mask or a mouthpiece. Other devices have been developed such as AERx (Aradigm, Hayward, Calif.) and Respimat (Boehringer, Germany) that generate an aerosol mechanically and vibrating mesh technologies such as AeroDose (Aerogen, Inc., Galway, Ireland), Eflow (Pari, Stanrberg, Germany) and MicroAir (Omron, Japan) nebulizers used to deliver proteins and peptide-based pharmaceuticals to the lungs. In every case the size of aerosolized particles should remain <5 μM to allow an adequate lung targeting.

In other embodiments, metered dose inhalers may be used. In yet other embodiments, dry powder delivery devices are also known and can be used.

The amount of cationic polymers as active ingredient which can be combined with a carrier material to produce a single dosage form will vary depending upon the subject being treated, and the particular mode of administration.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

In one specific embodiment, the pharmaceutical composition of the invention comprises said cationic polymers in an amount therapeutically effective to reduce at least one of the following: (i) the visco-elasticity of thick, mucoid or mucopurulent sputum in the airways of a patient, (ii) pathogens infectivity, (iii) inflammation and (iv) protease activity and/or to enhance anti-protease activity in cystic fibrosis sputa in vitro.

### The Cationic Polymers for Use as a Medicament

The cationic polymers according to the invention are predicted to be useful in (i) fluidizing lung secretions, (ii) protecting from infections and (iii) inflammation and (iv) stimulating activity of protease inhibitors, in patients suffering from accumulation of thick, mucoid or mucopurulent sputum in the airways.

Therefore, the cationic polymers according to the invention may be used as a medicament, in particular for the treatment of inflammatory lung disorders characterized by the accumulation of thick, mucoid or mucopurulent sputum in the airways.

Disorders characterized by accumulation of thick, mucoid or mucopurulent sputum in the airways includes, without limitation, cystic fibrosis, chronic bronchitis, infectious pneumonia, chronic obstructive lung/pulmonary disease (COLD/COPD), asthma, tuberculosis, fungal infections, airways manifestations of mucopolysaccharidoses I, II, IIIA, IIIB, IIIC, VI and VII and sinusitis.

“Treatment” is herein defined as the application or administration of cationic polymers according to the invention, or a pharmaceutical composition comprising said cationic polymers, preferably as an aerosol, into the airways, to a subject, where the subject has a disorder or a symptom associated with accumulation of thick, mucoid or mucopurulent sputum in the airways, where the purpose is to cure, heal, alleviate, relieve, alter, remedy, ameliorate, improve said inflammatory lung disorder, or any symptom associated to said disorder, in particular mucociliary clearance.

By “positive therapeutic response” with respect to said inflammatory lung disorders, such as cystic fibrosis, is intended an improvement in the disease in association with the mucus fluidizing activity of these molecules or compositions according to the invention, and/or their stimulatory effect on protease inhibitors, and/or an improvement in any of the symptoms associated with the disease.

The effect of mucus fluidizing activity can be measured by monitoring visco-elasticity of a sputum sample from the patient prior to and after treatment with the compositions according to the invention, using in vitro assays (viscosity measurement) as described in the Examples.

By “therapeutically effective dose or amount” or “effective amount” is intended to be an amount of cationic polymers of the invention, or a pharmaceutical composition according to the invention, that, when administered brings about a positive therapeutic response with respect to treatment of a subject with a lung disorder characterized by the recruitment of blood neutrophils in the airways favouring formation of a thick, mucoid or mucopurulent sputum (e.g. cystic fibrosis).

Since the cationic polymers of the invention have been shown to stimulate the in vivo inhibition of protease inhibitors in lung secretions of patients, in some embodiments of the invention, the cationic polymers of the present invention may be advantageously administered in combination with at least one protease inhibitor, wherein the cationic polymers and the other therapeutic agent(s) may be administered sequentially, in either order, or simultaneously (i.e., concurrently or within the same time frame).

Examples of suitable protease inhibitors that can be administered in combination with the cationic polymers of the invention, include but are not limited to the naturally occurring inhibitors of proteases, inhibitors of chelionanin family, α1-antitrypsin, antichymotrypin, serpins including serpin B1, secretory leukoprotease inhibitor, elafin and its precursor trappin-2. Recombinant, and chemical inhibitors that inhibit one, two or the three neutrophil serine proteases, can be used alone or in combination for in vivo administration.

α1-antitrypsin includes plasma-derived, glycosylated, human α1-antitrypsin, such as PROLASTIN®, ARALAST™, or ZAMAIRA™. Alternatively, compositions comprising recombinant α1-antitrypsin or any functional derivatives may be used.

In another specific embodiment, the cationic polymers are administered in combination with one or more additional active ingredients, especially, conventional active ingredients for the treatment of pulmonary disorders.

In specific embodiments, said additional active ingredients are selected from the group consisting of DNase, antibiotics, N-acetylcysteine, trypsin, chymotrypsin, glucocorticosteroids, amiloride triphosphate, uridine triphosphate, hypertonic saline, secretory leukoprotease inhibitor, bronchodilators, anti-inflammatory agents, mucolyitics and α1-antitrypsin.

The invention also relates to a method for reducing, in patients in need thereof, the visco-elasticity of thick, mucoid or mucopurulent sputum present in the airways of said patients, the method comprising the step of administering to the patient, at least the cationic polymers of the invention, or the pharmaceutical compositions, as defined above, in an amount therapeutically effective to reduce the visco-elasticity of said thick, mucoid or mucopurulent sputum.

The following examples are offered by way of illustration and not by way of limitation.

### LEGENDS OF THE FIGURES

FIG. 1: A/ variations of the apparent cell count in sputum homogenate after DNase and pLK treatment (results are expressed as a percentage of control; median±interquartiles) B/ Quantification of extracellular DNA in CF sputum effect after treatment with DNase and pLK (results are expressed as a percentage of control; median±interquartiles). pLK is pLK, HBr of 40,000-50,000 Da molecular weight.

FIG. 2: A/ Quantification of proteases activities in whole CF sputa before and after treatment by DNase, pLK or DNase+pLK showing the increase of elastase activity but not that of the other two proteases (median±interquartiles) B/ inhibition of proteases activities by α1-Pi (HNE and Pr3) and ACT (CG) before and after treatment by DNase, pLK or DNase+pLK. pLK is pLK, HBr of 40,000-50,000 Da molecular weight.

FIG. 3: A/ Extracellular DNA quantification in untreated or pLK- or PEG-g-pLK-treated sputum homogenates B/ Quantification of proteases activities in whole CF sputa before and after treatment by pLK or by PEG-g-pLK) (median±interquartiles) C/ inhibition of proteases activities by α1-Pi (HNE and Pr3) and ACT (CG) before and after treatment by pLK or PEG-g-pLK. pLK is pLK, HBr of 40,000-50,000 Da molecular weight. PEG-g-pLK is pLK, HBr of 40,000-50,000 Da molecular weight grafted with one mPEG molecule of 5,000 Da.

FIG. 4: A/ Influence of DNase and pLK on bacterial proliferation and wall permeabilization as assessed by DNA staining with a non-cell permeant fluorophore B/Scanning electron microscopy of bacteria after DNase and pLK treatment showing the spiculated surface of pLK-treated P aeruginosa indicating cell pemeabilization as described by (24). pLK is pLK, HBr of 40,000-50,000 Da molecular weight.

### EXAMPLES

### Materials and Methods

Patients: CF patients of the “Centre de Ressources et de Compétences de la Mucoviscidose” (CRCM) of Tours were included in the study and gave written informed consent. The inclusion criteria were a stable pulmonary disease, as defined by the clinical profile, and no hospitalization or change in their antibiotic and anti-inflammatory regimen during the month prior to inclusion. The research was carried out in accordance with the Helsinki Declaration (2000) of the World Medical Association and was approved by the local Ethical Committee (#2007-R17).

Sputum processing-CF sputum was collected into 50 ml Falcon® tubes by physiotherapy and processed immediately. Sputum was diluted with 3 volumes of PBS per gram and homogenized to obtain a crude homogenate that was kept on ice. An aliquot of each homogenate was incubated for 2 h in low-binding microtubes with 400 μg/ml DNase or with 1.5 mg/ml of poly-L-Lysine (pLK) or PEGylated pLK (PEG-g-pLK) under gentle stirring at room temperature, then layered on a glass slide for cell counting by trypan blue exclusion and visual aspect under the optical microscope. Different sizes of pLK were tested (pLK, HBr of either 500-2,000 Da, 1000-5,000 Da, 4,000-15,000 Da and 40,000-50,000 Da molecular weight). PEG-g-pLK was pLK of 40,000-50,000 Da molecular weight grafted with one mPEG molecule of 5,000 Da.

### Rheology and Viscosity Measurements

The viscoelastic properties of the mucus can be analysed by using a controlled stress rheometer equipped with a cone-plate geometry following standardized procedures (25).

### Quantification of Extracellular DNA

Extracellular DNA is quantified with the non-cell-permeant fluorochrome EvaGreen™ dsDNA reagent (λₑₓ=488 nm and λₑₘ=520 nm).

### Measurement of Peptidase Activities

Proteases activities were measured as described in (26) with the specific FRET (fluorescence resonance energy transfer) substrates of HNE, Pr3 and CG: Abz-APEEIMRRQ-YNO2, Abz-VADnVADQ-EDDnp and Abz-TPFSGQ-YNO2 respectively (λₑₓ=320 nm and λₑₘ=420 nm). The concentrations of active proteases in biological samples were determined by comparing the rates of hydrolysis of their specific substrates with those of commercial titrated proteases under the same experimental conditions. HNE and Pr3 were titrated as described in (27) and CG was titrated with HNE-titrated recombinant human secretory leukocyte protease inhibitor.

### Sputum Proteases Inhibition by Exogenous Inhibitors

Aliquots of sputum homogenates containing peptidases in the 10 nanomolar range, were incubated for 30 min at room temperature with increasing amounts of inhibitors up to a (I)/(E) molar ratio of about 40. The low Mr recombinant inhibitor EPI-hNE4 or α1-proteinase inhibitor (α1-Pi) were used to inhibit HNE, while α1-antichymotrypsin (ACT) was used to inhibit CG; Pr3 was inhibited with α1-Pi after samples had been incubated with a 1000-fold molar excess of EPI-hNE4 to avoid the α1-Pi interacting with HNE (8).

### Culture of Bacteria

S. aureus (strain CIP 103811) and P. aeruginosa (strain PAO1) were grown to exponential phase in brain heart infusion medium with aeration, collected by centrifugation at 10,000×g for 10 min at 20° C., washed and suspended in PBS. The bacterial count was determined by the measure of OD₆₀₀ₙₘ.

### Antimicrobial Tests

6×10⁶ bacteria were incubated in low-binding 96-wells microplates 3 h in 150 μl PBS containing 110 μg of DNase or 2 μg of pLK (MW 40,000-50,000 Da). Aliquots of the media were then collected and serial dilutions in PBS were laid on agar plates. CFUs were numbered after incubation at 37° C. Extracellular DNA in the remaining media was quantified as previously described as a marker of bacterial wall permeability.

### Confocal Microscopy

Aliquots of DNase-treated, pLK-rhodamine-treated (pLK of 40,000-50,000 Da) or untreated sputum homogenate were seeded onto Superfrost slides (CML, Nemours, France). Dry samples were washed in PBS to remove the excess of pLK-rhodamine and were then fixed by incubation in 4% (v/v) formaldehyde in PBS for 30 min at room temperature. After washing, dsDNA was detected by incubating the samples with 10 nM of the cell-permeant fluorochrome DRAQ5™ in PBS for 30 min. Finally, samples were washed, mounted with Fluoromount and examined under an Olympus FV 500 confocal microscope.

### Scanning Electron Microscopy

6×10⁶ bacteria were settled on polylysine-coated glass slides and treated (or not) with DNase or pLK (pLK of 40,000-50,000 Da). They were then fixed with 1% (v/v) glutaraldehyde and 4% (v/v) paraformaldehyde in 0.1 M PBS, pH 7.4, post-fixed in 2% (v/v) osmium tetroxide, dehydrated in a graded acetone series, dried to the critical point using carbon dioxide, and sputter coated with platinum. Fixed cells were examined with a Zeiss Gemini 982 scanning electron microscope.

### Results

### pLK Fluidizes CF Sputum:

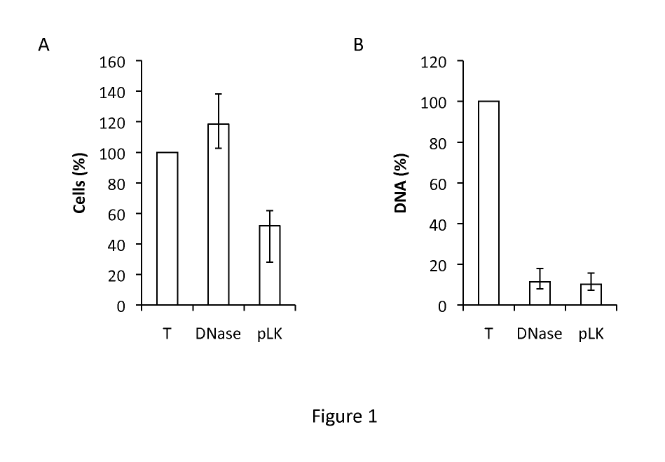

The increased viscosity of mucus in the CF lung leads to lung obstruction and subsequent decrease in lung function. Because DNA is a major component of CF lung secretions that contributes to their viscosity, rhDNase has been used for long by CF patients as a liquefying expectorant (28). A possible other mean to fluidize lung secretions could be through the condensation of extracellular DNA by positively charged polymers. We checked this hypothesis using poly-L-lysine (pLK) and comparing the rheological properties of CF sputa before and after treatment by pLK. Then we compared the results with those obtained using DNase in the same conditions. We observed no morphological change of the sputum after DNase treatment whereas pLK induced the fractionation of the sputum in dense aggregates and a very fluid phase (data not shown). Most of sputum neutrophils were found in these aggregates that explain an apparent decrease in cell counting (FIG. 1A).

We observed an important decrease in the amount of extracellular DNA after DNase treatment and also after incubation with pLK (FIG. 1B). But DNA was actually not degraded in pLK-treated sputum, since its extracellular content was restored after addition of dextran sulfate (Data not shown). It is well known that DNA condensation into dense aggregates impairs dye intercalation as ethydium bromide, propidium bromide including DRAQ5™ and visualization by confocal microscopy (Data not shown). This is supported by the observation of a more intense DNA labeling at the periphery of aggregates where DNA condensation is lower.

The formation of dense aggregates results in the formation of a liquid and almost transparent phase. Whether this may be helpful to improve mucociliary clearance in CF remains to be demonstrated.

### pLK Helps Improving Neutrophil Serine Proteases Inhibition in Sputum:

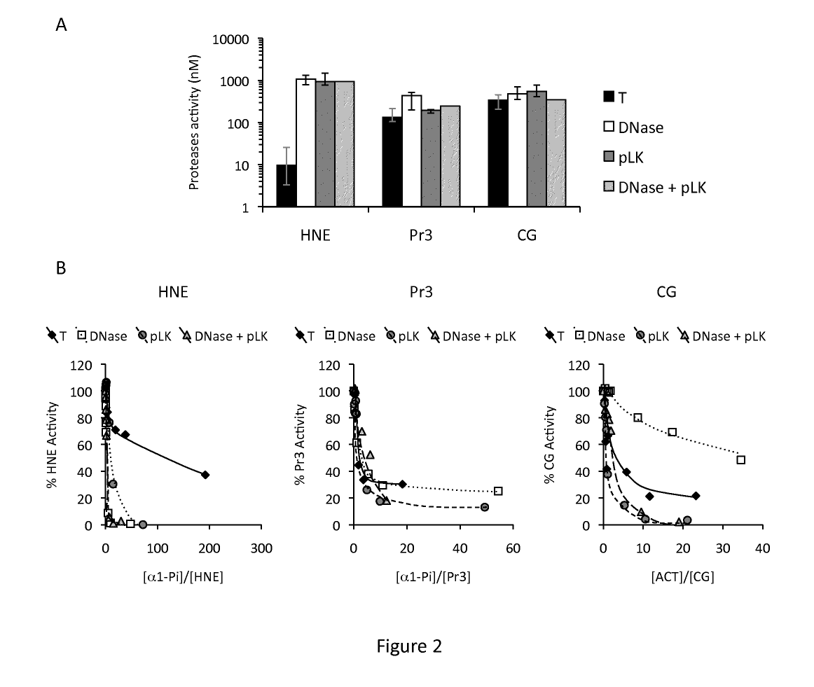

CF-associated chronic lung inflammation depends in part on a proteases/antiproteases imbalance resulting from the recruitment of blood neutrophils in the airways. Thus, a protease inhibitor-based therapeutic treatment could potentially help combating protease-dependant inflammation. But we previously showed that proteases resisted inhibition in CF sputum due to their binding to DNA and other negatively charged macromolecular components (8). DNase treatment of whole sputum induces a dramatic increase in elastase activity but this can be completely and stoichiometrically inhibited by elastase inhibitors (FIG. 2). We obtained the same result after compaction of DNA by pLK: elastase activity was stoichiometrically inhibited by α1-Pi after pLK treatment in spite of a dramatic increase in its activity in whole sputum (FIG. 2). Neither pLK nor DNase treatments significantly affected the activity and inhibition of PR3 by α1-Pi but pLK, unlike DNase, significantly improved CG inhibition by ACT (FIG. 2). This is due to the absence of DNA fragmentation after pLK treatment since the DNA fragments generated by DNase counteract interaction of CG with its inhibitors (8, 29); Using a mixture of pLK and DNase gave the same result as pLK alone which demonstrates that DNA compaction occurs more rapidly than DNA fragmentation and that compacted DNA resist degradation by DNase.

Thus the combined use of pLK and inhibitors of HNE and CG could help limiting uncontrolled proteolysis in the lung and the resulting inflammation.

Testing pLK of different sizes showed that pLK with a MW in the 4,000-15,000 Da range gave the same results as those with higher MW, whereas those with lower MW (500-2,000 Da and 1,000-5,000 Da) were far less efficient. Experiments are currently done to determine the optimal size of pLK combining good DNA condensation and low toxicity.

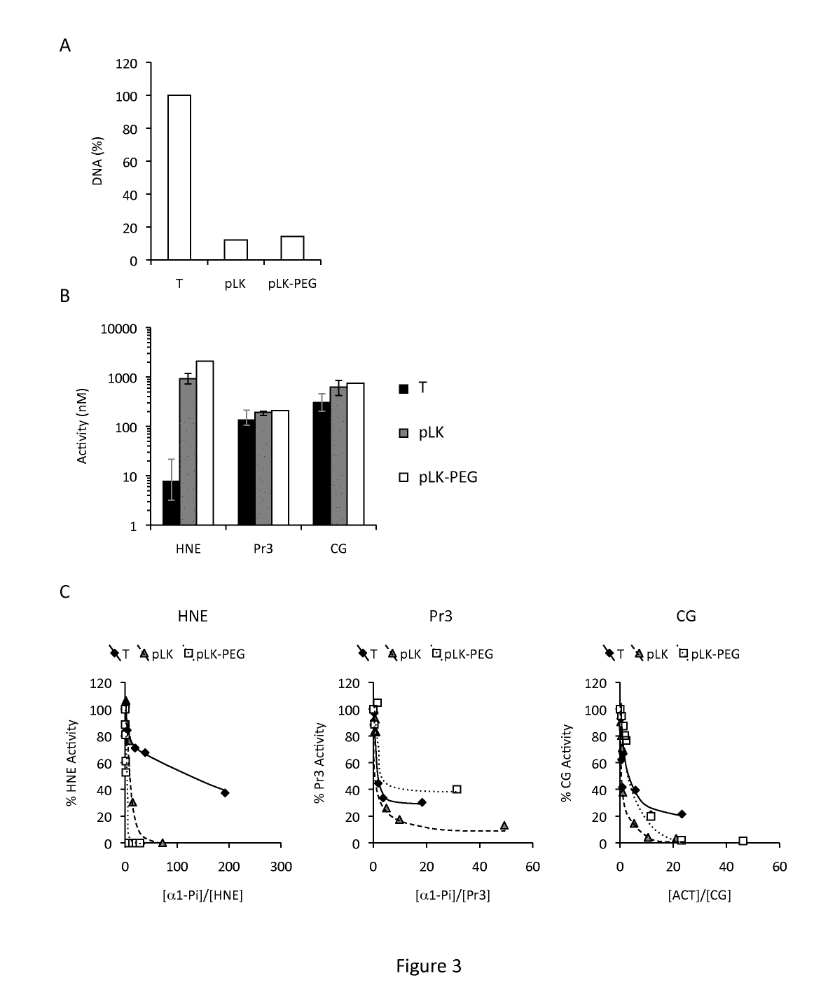

Because of the possible difficulty to remove large aggregates by mucociliary clearance, macrophages uptake or expectoration we tested a PEGylated pLK that decreases DNA/polycations complexes aggregation, interaction with plasma proteins, and cytotoxicity (30). Preliminary results show that the aggregates generated by PEG-g-pLK are less dense and viscous, and they regulate proteases activities in the same way as crude pLK (FIG. 3).

Microbicidal Properties of pLK Towards S. aureus and P. aeruginosa:

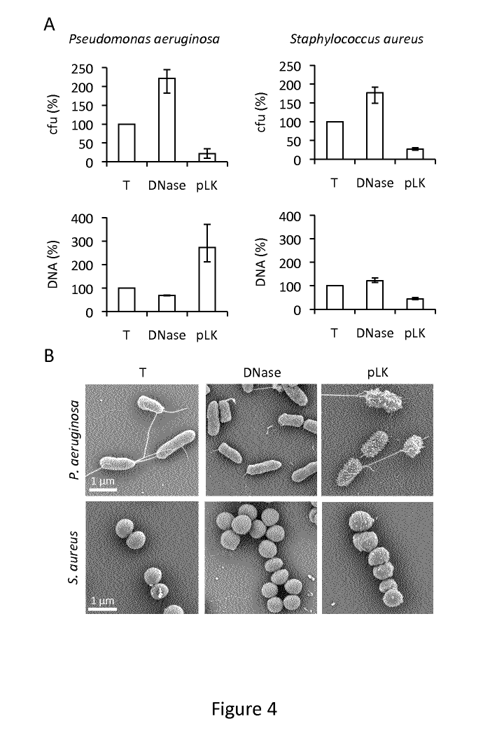

CF is characterized by persistent lung infections, especially by Staphylococcus aureus and Pseudomonas aeruginosa (1). Resistant strains of these bacteria that colonize the thick CF mucus impair antibiotics access and thus compromise their elimination from the lungs of contaminated patients. Because natural antimicrobial peptides and proteins act through their cationic charge (31), we looked at whether pLK possessed antimicrobial properties against bacterial cultures of S. aureus and P. aeruginosa. Indeed pLK displayed a significant bactericidal effect toward the two pathogens (FIG. 4A). This associates a significant modification of the morphology of P. aeruginosa as visualized by a spiculated cell surface (FIG. 4) and a disruption of the bacterial wall as quantified by the measure of extracellular DNA with a non-cell permeant fluorophore (FIG. 4A).

Morphological changes were less marked and no wall permeabilization was observed with S. aureus (FIG. 4B). Unlike pLK, DNase showed no antimicrobial properties nor it affected the morphology of the bacteria (FIG. 4). We conclude that in addition to its fluidizing properties pLK may control bacterial colonization of CF lungs by gram negative and gram-positive bacteria.

### Toxicological Studies

The objective of the study was to identify the nature and the dose of polycations to be aerosolized safely in control mice.

In vivo assessment of polycations toxicity has been done by cytologic analysis of bronchoalveolar lavage fluids (BALFs), quantification of proinflammatory cytokines (IL6 and KC) and lung anatomopathology.

Aerosol administration of different doses of pLK was done using a Microsprayer®. Mice were killed at day 2 and day 5 and bronchoalveolar lavage fluids (BALF) were obtained with instillation of 5×0.5 mL of sterile PBS into the lungs. Lungs were perfused with 4% formaldehyde for histological studies. Cells were collected after centrifugation of BALFs, suspended in PBS, and analyzed by flow cytometry. The supernatant was used for cytokine quantification using commercially available ELISA kits.

Remarkably, we observed no significant differences in BALF cellularity, and cytokines quantification between control and treated mice (Mann-Whitney test) in response to aerosol administration of 2.4 mg/kg pLK (4-15 kDa) or pLK(4-15 kDa)-PEG (66 μg pLK in 50 μL corresponding to a concentration of about 130 μM) for a mouse of 27-30 g. The histological study confirmed these observations by showing a minimal broncho-interstitial inflammation at day 2 both in treated and in control groups, but no lung lesion was observed at day 2 nor at day 5.

However, a cellular toxicity of pLK (4-15 kDa) or pLK(4-15 kDa)-PEG can be observed using in vitro cultures of Beas-2B cells. Interestingly, the in vitro toxicity threshold was lowered by a factor 20 (5 μM vs 100 μM) using pLK-His358PM (His 50%) whereas the antibacterial properties of pLK-His against P aeruginosa remained unchanged.

### REFERENCES

1. Hauser, A. R., Jain, M., Bar-Meir, M., and McColley, S. A. (2011) Clinical significance of microbial infection and adaptation in cystic fibrosis. Clin Microbiol Rev 24, 29-70
2. Voynow, J. A., and Rubin, B. K. (2009) Mucins, mucus, and sputum. Chest 135, 505-512
3. Brinkmann, V., and Zychlinsky, A. (2007) Beneficial suicide: why neutrophils die to make NETs. Nat Rev Microbiol 5, 577-582
4. Manzenreiter, R., Kienberger, F., Marcos, V., Schilcher, K., Krautgartner, W. D., Obermayer, A., Huml, M., Stoiber, W., Hector, A., Griese, M., Hannig, M., Studnicka, M., Vitkov, L., and Hartl, D. (2011) Ultrastructural characterization of cystic fibrosis sputum using atomic force and scanning electron microscopy. J Cyst Fibros
5. Suri, R. (2005) The use of human deoxyribonuclease (rhDNase) in the management of cystic fibrosis. BioDrugs 19, 135-144
6. Donaldson, S. H., Bennett, W. D., Zeman, K. L., Knowles, M. R., Tarran, R., and Boucher, R. C. (2006) Mucus clearance and lung function in cystic fibrosis with hypertonic saline. N Engl J Med 354, 241-250
7. Griese, M., Kappler, M., Gaggar, A., and Hartl, D. (2008) Inhibition of airway proteases in cystic fibrosis lung disease. Eur Respir J 32, 783-795
8. Dubois, A. V., Gauthier, A., Brea, D., Varaigne, F., Diot, P., Gauthier, F., and Attucci, S. (2012) Influence of DNA on the activities and inhibition of neutrophil serine proteases in cystic fibrosis sputum. Am J Respir Cell Mol Biol
9. Park, T. G., Jeong, J. H., and Kim, S. W. (2006) Current status of polymeric gene delivery systems. Adv Drug Deliv Rev 58, 467-486
10. Shima, S., Matsuoka, H., Iwamoto, T., and Sakai, H. (1984) Antimicrobial action of epsilon-poly-L-lysine. J Antibiot (Tokyo) 37, 1449-1455
11. Scholz, C., and Wagner, E. (2011) Therapeutic plasmid DNA versus siRNA delivery: Common and different tasks for synthetic carriers. J Control Release
12. Ogris, M., Brunner, S., Schuller, S., Kircheis, R., and Wagner, E. (1999) PEGylated DNA/transferrin-PEI complexes: reduced interaction with blood components, extended circulation in blood and potential for systemic gene delivery. Gene Ther 6, 595-605
13. Toncheva, V., Wolfert, M. A., Dash, P. R., Oupicky, D., Ulbrich, K., Seymour, L. W., and Schacht, E. H. (1998) Novel vectors for gene delivery formed by self-assembly of DNA with poly(L-lysine) grafted with hydrophilic polymers. Biochim Biophys Acta. 1380, 354-368
14. Choi, Y. H., Liu, F., Choi, J. S., Kim, S. W., and Park, J. S. (1999) Characterization of a targeted gene carrier, lactose-polyethylene glycol-grafted poly-L-lysine and its complex with plasmid DNA. Hum Gene Ther. 10, 2657-2665
15. Dailey, L. A., Kleemann, E., Merdan, T., Petersen, H., Schmehl, T., Gessler, T., Hanze, J., Seeger, W., and Kissel, T. (2004) Modified polyethylenimines as non viral gene delivery systems for aerosol therapy: effects of nebulization on cellular uptake and transfection efficiency. J Control Release 100, 425-436
16. Mockey, M., Bourseau, E., Chandrashekhar, V., Chaudhuri, A., Lafosse, S., Le Cam, E., Quesniaux, V. F., Ryffel, B., Pichon, C., and Midoux, P. (2007) mRNA-based cancer vaccine: prevention of B16 melanoma progression and metastasis by systemic injection of MART1 mRNA histidylated lipopolyplexes. Cancer Gene Ther 14, 802-814
17. Ziady, A. G., Gedeon, C. R., Miller, T., Quan, W., Payne, J. M., Hyatt, S. L., Fink, T. L., Muhammad, 0., Oette, S., Kowalczyk, T., Pasumarthy, M. K., Moen, R. C., Cooper, M. J., and Davis, P. B. (2003) Transfection of airway epithelium by stable PEGylated poly-L-lysine DNA nanoparticles in vivo. Mol Ther 8, 936-947
18. Itaka, K., Osada, K., Morii, K., Kim, P., Yun, S. H., and Kataoka, K. (2010) Polyplex nanomicelle promotes hydrodynamic gene introduction to skeletal muscle. J Control Release 143, 112-119
19. Erbacher, P., Roche, A. C., Monsigny, M., and Midoux, P. (1995) Glycosylated polylysine/DNA complexes: gene transfer efficiency in relation with the size and the sugar substitution level of glycosylated polylysines and with the plasmid size. Bioconjug Chem 6, 401-410
20. Erbacher, P., Bousser, M. T., Raimond, J., Monsigny, M., Midoux, P., and Roche, A. C. (1996) Gene transfer by DNA/glycosylated polylysine complexes into human blood monocyte-derived macrophages. Hum Gene Ther 7, 721-729
21. Sagara, K., and Kim, S. W. (2002) A new synthesis of galactose-poly(ethylene glycol)-polyethylenimine for gene delivery to hepatocytes. J Control Release 79, 271-281
22. Erbacher, P., Roche, A. C., Monsigny, M., and Midoux, P. (1997) The reduction of the positive charges of polylysine by partial gluconoylation increases the transfection efficiency of polylysine/DNA complexes. Biochim Biophys Acta 1324, 27-36
23. Berge, S. M., Bighley, L. D., and Monkhouse, D. C. (1977) Pharmaceutical salts. J Pharm Sci 66, 1-19
24. Hilpert, K., Elliott, M., Jenssen, H., Kindrachuk, J., Fjell, C. D., Korner, J., Winkler, D. F., Weaver, L. L., Henklein, P., Ulrich, A. S., Chiang, S. H., Farmer, S. W., Pante, N., Volkmer, R., and Hancock, R. E. (2009) Screening and characterization of surface-tethered cationic peptides for antimicrobial activity. Chem Biol 16, 58-69
25. Zahm, J. M., Girod de Bentzmann, S., Deneuville, E., Perrot-Minnot, C., Dabadie, A., Pennaforte, F., Roussey, M., Shak, S., and Puchelle, E. (1995) Dose-dependent in vitro effect of recombinant human DNase on rheological and transport properties of cystic fibrosis respiratory mucus. Eur Respir J 8, 381-386
26. Korkmaz, B., Attucci, S., Juliano, M. A., Kalupov, T., Jourdan, M. L., Juliano, L., and Gauthier, F. (2008) Measuring elastase, proteinase 3 and cathepsin G activities at the surface of human neutrophils with fluorescence resonance energy transfer substrates. Nat Protoc 3, 991-1000
27. Korkmaz, B., Poutrain, P., Hazouard, E., de Monte, M., Attucci, S., and Gauthier, F. L. (2005) Competition between elastase and related proteases from human neutrophil for binding to alpha1-protease inhibitor. Am J Respir Cell Mol Biol 32, 553-559
28. Hodson, M. E., and Shah, P. L. (1995) DNase trials in cystic fibrosis. Eur Respir J 8, 1786-1791
29. Duranton, J., Boudier, C., Belorgey, D., Mellet, P., and Bieth, J. G. (2000) DNA strongly impairs the inhibition of cathepsin G by alpha(1)-antichymotrypsin and alpha(1)-proteinase inhibitor. J Biol Chem 275, 3787-3792
30. Lv, H., Zhang, S., Wang, B., Cui, S., and Yan, J. (2006) Toxicity of cationic lipids and cationic polymers in gene delivery. J Control Release 114, 100-109
31. Hancock, R. E., and Diamond, G. (2000) The role of cationic antimicrobial peptides in innate host defences. Trends Microbiol 8, 402-410

1. A pharmaceutical composition comprising a cationic polymer in combination with at least a protease inhibitor.

2. The pharmaceutical composition of claim 1, wherein said protease inhibitor is selected from the group consisting of: serpins, natural inhibitors of the chelonianin family, α1-antitrypsin, antichymotrypin, serpin B1, secretory leukoprotease inhibitor and elafin.

3. The pharmaceutical composition of claim 1, wherein said cationic polymer is present in an effective amount for enhancing anti-protease activity of said protease inhibitor in cystic fibrosis sputa in vitro.

4. The pharmaceutical composition of claim 1, wherein said cationic polymer has at least 24 positively charged monomeric units.

5. The pharmaceutical composition of claim 4, wherein said positively charged monomeric units are selected from the group consisting of positively charged amino acids.

6. The pharmaceutical composition of claim 5, wherein said cationic polymer is a polyaminoacid with a molecular weight between 4 and 15 kDa.

7. The pharmaceutical composition of claim 5, wherein said positively charged amino acids are selected from the group consisting of lysine, arginine, histidine or ornithine, for example the cationic polymer is poly-L-lysine.

8. The pharmaceutical composition of claim 1, wherein said cationic polymer is further PEGylated and/or glycosylated and/or gluconoylated.

9. The pharmaceutical composition of claim 1, which is an aerosol.

10. A method for treating an inflammatory lung disorder characterized by the recruitment of blood neutrophils in the airways favoring the formation of a thick, mucoid or mucopurulent sputum in a subject in need thereof, comprising
administering to the subject a therapeutically efficient amount of a pharmaceutical composition of claim 1.

11. A method for treating, in a subject in need thereof, an inflammatory lung disorder characterized by the recruitment of blood neutrophils in the airways favoring the formation of a thick, mucoid or mucopurulent sputum, comprising
administering to the subject a composition comprising i) a cationic polymer in an amount therapeutically effective to reduce the visco-elasticity of the sputum, and ii) a pharmaceutically acceptable excipient.

12. The method of claim 11, wherein said cationic polymer has at least 24 positively charged monomeric units.

13. The method of claim 11, wherein said cationic polymer is a polyaminoacid with at least 70% of positively charged amino acids with a molecular weight between 4 and 15 kDa.

14. The method of claim 13, wherein said charged amino acids are selected from the group consisting of lysine, arginine, histidine and ornithine.

15. The composition method of claim 11, wherein said composition does not comprise any nucleic acid molecule.

16. The composition of claim 4, wherein said cationic polymer has between 30 and 72 positively charged monomeric units

17. The composition of claim 1, wherein said cationic polymer is poly-L-lysine.

18. The method of claim 10, wherein said inflammatory lung disorder is cystic fibrosis.

19. The method of claim 11, wherein said inflammatory lung disorder is cystic fibrosis.

20. The pharmaceutical composition of claim 1, wherein said cationic polymer is polylysine partially substituted with histidine residues and/or neutral residues.

21. The pharmaceutical composition of claim 20, wherein no more than 50% of the lysyl residues in said polylysine are substituted with said histidine residues and/or said neutral residues.

22. The method of claim 11, wherein said cationic polymer is polylysine partially substituted with histidine residues and/or neutral residues.

23. The method of claim 22, wherein no more than 50% of the lysyl residues in said polylysine are substituted with said histidine residues and/or said neutral residues.
